(19) United States
(12) Patent Application Publication
(10) Pub. No.: US 2009/0004212 A1
(43) Pub. Date: Jan. 1, 2009

(54) Tumour vaccines for MUC1-positive carcinomas

(76) Inventor: Franz-Georg Hanisch, Koln (DE)

Correspondence Address: BUCHANAN INGERSOLL & ROONEY PC, P.O. BOX 1404, ALEXANDRIA, VA, 22313-1404, US

(21) Appl. No.: 12/008,733

(22) Filed: Jan. 14, 2008

Related U.S. Application Data
(63) Continuation-in-part of application No. 09/606,910, filed on Jun. 29, 2000, now Pat. No. 7,342,094.
Application No. 09/606,910 is a continuation of application No. PCT/DE98/03819, filed on Dec. 30, 1998.

(30) Foreign Application Priority Data
Dec. 30, 1997 (DE) 19758400.4

(51) Int. Cl. A61K 39/00; C07K 1/107; C12P 21/02; A61K 38/14; A61K 35/12; C12N 5/06

(52) U.S. Cl. 424/185.1; 530/322; 435/68.1; 514/8; 424/93.7; 435/325

(57) ABSTRACT

Embodiments of the invention provide tumour vaccines, especially for activation of glycopeptide-specific cytotoxic T-cells by MHC class I pathway, comprising at least one peptide of 8-11 amino acids derived from the region SAPDTRPAPGST of the human epithelial mucin MUC1 containing the immunodominant PDTRPAP region and which is glycosylated on threonine of the immunodominant PDTRPAP region and start with SAP, APD or PDT at it's N-terminus.

The invention relates to tumour vaccines of a new type which are based on the molecular structure of human epithelial mucin (MUC1). The invention can be used for the immunotherapy of carcinomas.

Epithelial mucins are glycoproteins with repetitive amino acid sequences and a high proportion of carbohydrates which are partially bound to membranes, partially secreted and are to be found on many glandular epithelia. The epithelial mucin known best is the membrane-bound MUC1, described also as PEM, PUM, EMA, MAM-6, PAS-0 or episialine (Finn, O. et al., Immunol. Reviews 145:61, 1995) the extracellular part of which consists of a variable number of repeating units of 20 amino acids, the so-called “tandem repeats”. The MUC1 is not a tumour specific molecule per se; its suitability as tumour antigen is based on the fact that its carbohydrate portion is qualitatively and quantitatively changed in tumours (Burchell, J. and Taylor-Papadimitriou, J., Epith. Cell Biol. 2:155, 1993). Here, new epitopes appear which are detected by the immune system (humoral and cellular defence). After removing the primary tumour operatively (or after a radiation or chemotherapy) we have, as a rule, to proceed on the fact that tumour cells still remain in the body (“minimal residual disease”). These tumour cells which represent a potential danger are combated by various endogenic mechanisms the efficiency of which may be intensified by an adjuvant immunotherapy. The most effective adjuvant immunotherapy is vaccination. Here, two prerequisites are required: firstly, a suitable target antigen (epitope) shall be present on the tumour cells and secondly that it will be possible to prepare a form of vaccines as strongly immunogenically as possible, preferably a synthetic form.

The MUC1 glycoprotein is considered a tumor antigen due to its over-expression and aberrant glycosylation in cancer tissues. Effective immunotherapeutic anti-cancer strategies toward MUC1-positive carcinomas should activate MUC1 glycopeptide-specific cytotoxic T-cells. This presumes that ectodomain epitopes of MUC1 glycoforms can be presented to T-cells by the MHC class I system via cross-presentation pathways.

To specifically activate cytotoxic T-lymphocyte (CTL) clones reactive to ectodomain epitopes of a transmembrane glycoprotein these glycosylated epitopes need to enter the endoplasmic reticulum of antigen-presenting cells (APCs) via one of several cross-presenting mechanisms and be bound to and presented by MHC class I proteins on the surface of APCs. It is current knowledge that peptides bind into the binding cleft through their appropriately positioned side chains (anchor residues). For the human A2 allele (HLA-A*0201) binding peptides these anchors are located at peptide positions 2 and 9 (P2 and P9) with other peptide side chains, especially at P3, contributing to the stability of the complex (1).

Most HLA-A2-binding peptides are nine amino acid (aa) residues long, with Leu at P2, and Val or Leu at P9, but in certain cases the optimal length for peptide binding to MHC class I can be longer than nine residues (2). Presentation of glycopeptides by MHC class I still remains a largely unexplored topic. Only glycoproteins with a cytosolic or nuclear topology that carry O-GlcNAc modifications have been found presented as glycopeptides on the surface of APCs (3). No mucin-type O-glycoproteins were found presented via the MHC-l pathway, yet.

The reason for this could be related to the topology of these O-glycoproteins, which are confined to the compartments of the secretory pathway and to the extra-cellular surface of the plasma membrane, where the glycosylated ectodomains are inaccessible to the cytosolic processing machinery. Secreted or plasmamembrane-shed O-glycoproteins can be endocytosed by APCs and committed to cross-presentation pathways, which mediate proteolysis of these antigens and their presentation on MHC class I molecules (4-7). In one of the alternativ pathways the endosomal antigen escapes into the cytosol and gets access to the processing active immunoproteasomes (8, 9). In another experimentally supported scenario, endosomal cathepsins are involved in the processing (10, 11). We have recently obtained in vitro evidence that enzymes involved in either of the alternative cross-priming mechanisms are able to process O-glycosylated proteins (12, 13). More specifically, cytosolic immunoproteasomes and cathepsin-L as a major processing enzyme in the late endosomes are able to cleave O-glycosylated peptides of the MUC1 tandem repeat domain. The cleavage efficiency was found to be highly dependent on the sites of peptide modification, since both, immunoproteasomes and cathepsin L, showed a motif or site specificity of cleavage.

It has previously been recognized that the intensity of CTL responses is inversely correlated with the degree of MUC1 glycosylation (14) indicating that either the processing or later steps in the immunological cascade are affected by the modification. A likely explanation could be the prevention of binding to MHC class I molecules by the bulky sugar chains. In studies on MHC class II-glycopeptide interaction it was demonstrated that the glycans could be tolerated, if they are appropriately positioned, i.e. do not substitute anchor positions within the peptide (15). In these cases the glycans pointed away from the binding groove of the MHC class II molecule enabling interactions with glycopeptide-specific receptors of T-cells. In the context of MHC class I, only one study reported binding of the MUC1 glycopeptide to MHC class I molecules and revealed the potential interaction of glycans with the MHC protein. Apostolopoulos et al. could show that anoctamer of the MUC1 tandem repeat, glycosylated with a GalNAc residue at the central DTR motif, had much higher binding affinity to the mouse allele H-2 Kb than the corresponding nonglycosylated peptide. The authors were able to demonstrate moreover that this enhancement in binding activity was caused by the interaction of the P5-Thr-GalNAc residue with the C pocket(16).

Several attempts to stimulate MUC1 cancer-specific immune response by vaccination are reported, but did not provide evidence for sufficient eradication of cancer cells. These attempts were mostly directed toward non-glycosylated sections of the MUC1 repeat domain that are not presented in this form on tumor cells. On the contrary, the tumorassociated glycoforms of MUC1 express glycans at much higher density than normal glycoforms (e.g., lactation-associated MUC1). It is to rationalize, that non-glycosylated MUC1 is presented via the ERAD pathway in the neonatal thymus with the consequence that high affinity CTLs that would be self-reactive are depleted. Accordingly, attempts to stimulate a CTL response to nonglycosylated MUC1 should have a restricted efficacy. Moreover, even a response to the nonglycosylated target would be expected to have limited cytotoxic efficacy due to glycosylation of MUC1 on the tumor cell.

Successful attempts were made to break tolerance to MUC1 in human MUC1 transgenic mice and to induce B-cell responses to glycosylated epitopes (17). However, no studies were reported so far that intended the induction of cytotoxic T-cell responses in hMUC1 transgenic mice. Recently, Tn-(GalNAc-) glycopeptides of MUC1 were shown to elicit effective cytotoxic responses in HLA-A*0201 transgenic mice (18). However, the elicited CTLs did not induce selective lysis of human MUC1-expressing murine cell lines, probably due to low abundance or differently glycosylated epitopes on the target cells.

Therefore, a response against Glyco-MUC1-bearing cells is an important basis for finding promising antigens for vaccines for an effective immunotherapy of MUC1+ tumors. The induction of cytotoxic T-cells with an immunological specifity against Glyco-MUC1-bearing cells and which are able to lyse them, was not reported so far and especially not the activation of those glycopeptide-specific cytotoxic T-cells via the MHC class I pathway.

The invention is aimed at developing further tumour vaccines on the basis of the molecule structure of human epithelial mucin MUC1 suited for combating tumour cells which remained in the body after other therapies.

### BRIEF DESCRIPTION OF THE SEVERAL VIEWS OF THE DRAWINGS

Table 1 shows a number of fragments generated by immunoproteasomes and average fragment length in the digest of MUC1 repeat peptides and glycopeptides.

Table 2 shows relative amounts of 8- to 11-meric (A) and glycosylated proteolytic fragments(B).

Table 3 shows relative frequencies of 8- to 11-meric proteolytic fragments grouped according to their N-terminus.

Table 4 shows peptides eluted from exosomal MHC molecules after cross-presentation of MUC1 100-mer in a murine dendritic cell line.

Table 5 shows HLA-A*0201 allele binding prediction of proteasomal products.

FIG. 1 shows the in vitro digestion of non-glycosylated 100-mer and chromatography of the reaction products by HPLC.

FIG. 2 presents the ligation strengths of tested peptides compared with a negative control considered as 0% and positive control considered as 100%.

FIG. 3, FIG. 4, FIG. 5, and FIG. 6 show results of HLA/SAP10 Modeling.

FIG. 7 shows results of tests of responder cells for cytotoxic activity.

The task of the invention is solved according to claim 1, the subclaims are preferential variants.

With the peptides as well as the method according to the invention it is now possible to isolate cytotoxic T-cells specific for Glyco-MUC1-bearing tumor cells and to develop further tumor vaccines.

The present invention bases on the unexpected finding, that human immunoproteasomes and cathepsin-L are able to generate 8- to 11-meric fragment peptides and glycopeptides of the MUC1 tandem repeat domain in vitro and that the patterns of cleavage closely resemble those found for exosomal MHC-bound peptides after pulsing of mouse dendritic cells. The peptides

### 1) SAPDT(-GalNAc)RPAPG;

### 2)SAPDT(-Galβ-1, 3-GalNAc)RPAPG;

### 3) SAPDT(-GalNAc)RPAP;

### 4) SAPDT(-Galβ-1, 3-GalNAc)RPAP;

### 5) APDT(-GalNAc)RPAPG;

### 6) APDT(-Galβ-1, 3-GalNAc)RPAPG;

### 7) APDT(-GalNAc)RPAPGS;

### 8) APDT(-Galβ-1, 3-GalNAc)RPAPGS;

### 9) PDT(-GalNAc)RPAPGS;

### 10) PDT(-Galβ-1, 3-GalNAc)RPAPGS;

### 11) PDT(-GalNAc)RPAPGST;

12) PDT(-Galβ-1, 3-GalNAc)RPAPGST are claimed to bind to frequent class I alleles (HLA-A2) and to activate glycopeptide-specific cytotoxic T-cells, and to enable them to target tumor cells presenting those peptides.

Surprisingly, those peptides were found to bind equally strong to the MHC class I allele HLA-A*0201 as the non-glycosylated peptide. Computer modelling of the HLA/glyco peptide complexes revealed that the binding capacity of the glycopeptides might result from orientation of the sugar moiety into the MHC groove as previously described for the murine H-2 Kb allele.

Further those glycopeptides are able to prime specific CTL that are similarly effective in their cytotoxic activity as the unglycosylated corresponding peptides-primed CTL and hence represent another antigenic epitope usable in vaccines to target MUC1+ tumor cells.

Contrary to the general consent, that glycosylation of protein antigens represent a hurdle to effective processing and presentation for CTL activation, it could be shown, that glycopeptides are presented as MHC-bound epitopes and recognized by glycopeptide-specific CTLs. The immunological inertness of glycopeptides is largely dependent on the position of a glycosylation site within the peptide, by the type of glycan attached and by the orientation of peptide and glycan moieties within the MHC groove.

It could be shown, that glycosylated MUC1 tandem repeat peptides are effectively degraded by immunoproteasomes and by cathepsin-L. While multiply O-glycosylated peptides are poor processing substrates, singly substituted peptides were effectively degraded to 8- to 11-meric fragments. Localization of the sugar had no influence on the degree of fragment diversity or on the distribution of fragment lengths. However, fragment diversity was increased for glycosylated substrates in comparison to non-glycosylated. Most of the generated 8- to 11-meric fragments are non-glycosylated, except for antigenic substrates, where glycosylation is located at the DTR motif and at some distance from the major processing sites within the SAP and GST motifs. Thus, MUC1 glycosylation resulted in generation of a unique set of glycosylated peptides that potentially may fit the class I MHC binding groove.

To identify further effects of glycosylation on MHC class I epitope generation, it is determined whether the epitope glycosylation affects binding to HLA-A0201 on the surface of the TAPdeficient T2 cell line. Our results confirm that glycans at anchor residues necessary for MHC binding—as found e.g. in ST(-GalNAc)APPAHGVT, ST(-GalNAc)APPAHGV, and S(-GalNAc)APDTRPAPG strongly reduce binding affinity in comparison with the corresponding nonglycosylated peptide. Interestingly, the SAP10 peptide glycosylated with GalNAc at Thr5 [SAPDT(GalNAc)RPAPG] as an example, shows strong binding to HLA-A0201 via MHC stabilization assay, which is similar to binding activity of the non-glycosylated SAP10. This result is confirmed by the in vitro induction of specific CTLs that recognize and efficiently kill T2 target cells, which present SAP10 or SAP10-T5* peptides in their HLA-A2-restricted MHC groove.

Our molecular modelling of the HLA/SAP 10 complexes already suggests that the glycopeptide glycosylated at T5 with GalNAccould is bound into the HLA-A2 binding groove equally or even better than the non-glycosylated peptide, when the sugar moiety is oriented into the MHC groove as described for the H-2 Kb molecule. The buried GalNAc residue and the structural similarities of the exposed side-chains of the glycosylated and non-glycosylated SAP10 bound to HLA-A2 suggest a possible cross-reactivity of these peptides in the T-cell recognition. If the sugar is oriented out of the groove, the glycopeptide binding is predicted to be weaker than for the non-glycosylated peptide. The slightly weaker binding of the SAP10-T5-GalNAc that is observed in our experiments, indicate a mixed population of the glycopeptide conformers bound to MHC with sugar moiety both directed into and out of the HLA-A2 groove. Similar results are reported for binding to murine MHC molecules by other groups.

Generation of tumor-specific CTLs includes three main steps: 1) the ability of proteasomes to generate appropriately sized peptide epitopes, 2) the ability of MHC class I molecules to bind them, and 3) the presence of specific CTL precursors that should be stimulated by epitope-MHC molecules and costimulatory molecules. Although it is shown that peptides of 8-11 amino acids derived from the region SAPDTRPAPGST of the human epithelial mucin MUC1 which contain the immunodominant PDTRPAP region and which are glycosylated on threonine of this immunodominant PDTRPAP region and start with SAP, APD or PDT at it's N-terminus are able to activate glycopeptide-specific cytotoxic T-cells, especially by MHC class I pathway and represent therefore promising antigens for vaccines that elicit CTL for immunotherapy of MUC1+ tumors. Peptides can be glycosylated by an mono- or an disaccharide. Possible are all mucin typical glycosylations, which are initiated by GalNAc.

Tumor vaccines according to the invention preferably comprise peptides with a length of 9-10 amino acids and which are glycosylated with α-acetylgalactosamine (GalNAc) or Galβ-1, 3-GalNAc.

Suitable peptides are e.g.

### SAPDT(-GalNAc)RPAPG;

### SAPDT(-Galβ-1, 3-GalNAc)RPAPG;

### SAPDT(-GalNAc)RPAP;

### SAPDT(-Galβ-1, 3-GalNAc)RPAP;

### APDT(-GalNAc)RPAPG;

### APDT(-Galβ-1, 3-GalNAc)RPAPG;

### APDT(-GalNAc)RPAPGS;

### APDT(-Galβ-1, 3-GalNAc)RPAPGS;

### PDT(-GalNAc)RPAPGS;

### PDT(-Galβ-1, 3-GalNAc)RPAPGS;

### PDT(-GalNAc)RPAPGST;

### PDT(-Galβ-1, 3-GalNAc)RPAPGST.

The invention further relates to a therapeutic composition comprising a therapeutical effective amount of the described glycopeptides optionally in combination with an APC's pulsed with a peptid according to the invention and/or with a pharmaceutically acceptable carrier.

A suitable pharmaceutically acceptable carriers are heat shock proteins as well as covalently bound peptids, which allow receptor-mediated endocytosis by dendritic cells (DC's) or accelerated unspecific absorption across plasma membrane into cytosol of dendritic cells.

Hereinafter the invention will be explained in greater detail by an example.

In the current study, human immunoproteasomes and cathepsin-L as tools for the production of MHC class I binding peptides and glycopeptides corresponding to sections of the human MUC1 tandem repeat domain are used.

### EXAMPLE

### Cells

The DC2.4 cell line was obtained from the American Type Culture Collection and grown in DMEM medium supplemented with 10% FCS, penicillin/streptomycin (200 IU/ml-200 μg/ml), 1% nonessential amino acids, 50 μM 2-mercaptoethanol at 37° C. and 5% CO2. Cells were detached by 10 min incubation in PBS containing 2 mM EDTA, centrifuged at 180×g/5 min and split 1:10.

In all experimental procedures followed by mass spectrometric analyses, the cultivation of cells was carried out in media without phenol red.

The T2 cell line is a negative mutant for “Transporter associated with antigen processing” (TAP protein), expressing an empty HLA-A0201 allele of the MHC class I molecule on the cell surface.

The cell line was a kind gift from Prof. Jonathan Howard and Dr. Michael Knittler, University of Cologne, and was initially grown at 37° C. and 7.5% CO2 in IMDM media supplemented with 10% FCS and penicillin/streptomycin (200 IU/ml-200 μg/ml). For the MHC class I stabilization experiments, T2 cells were slowly adapted to reduced serum conditions: 2% FCS. For T-cell assays T2 cell line was kindly provided by Prof. Walter J. Storkus, University of Pittsburgh (PA, USA) and grown in RPMI1640 medium supplemented with 10% FBS, 2 mM L-glutamine, 1% nonessential amino acids, 100 IU/mL penicillin and 100 μg/mL streptomycin at 37° C. and 6% CO2.

### Peptides and Glycopeptides

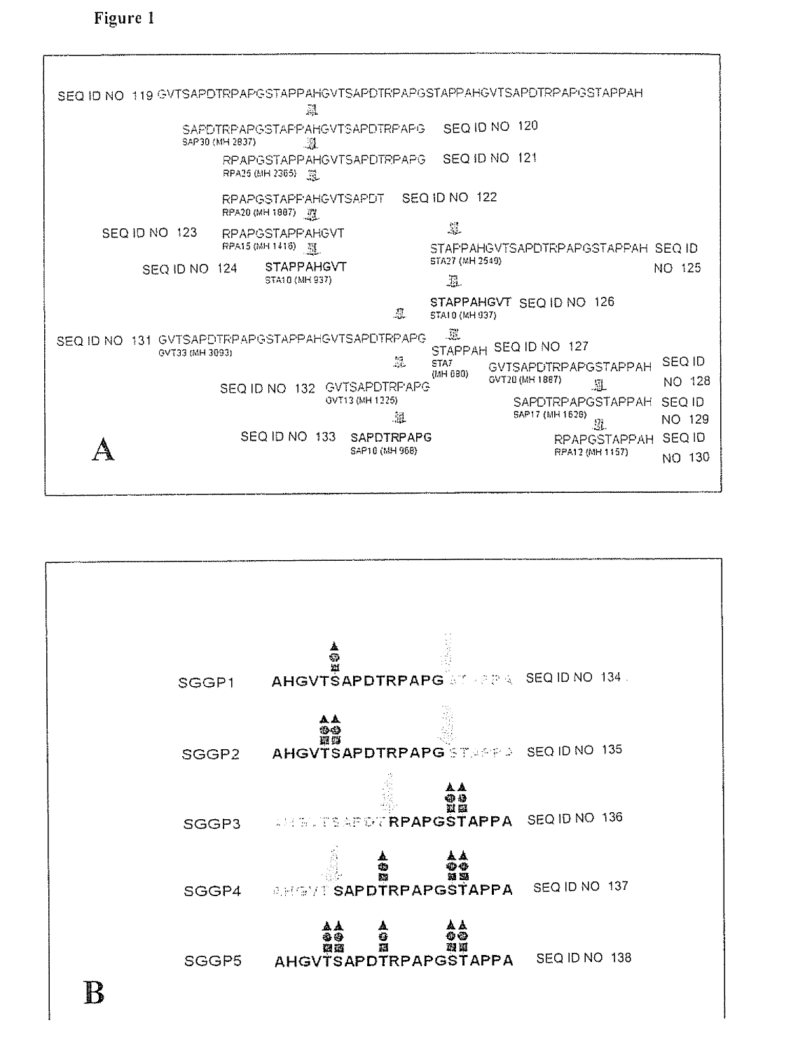

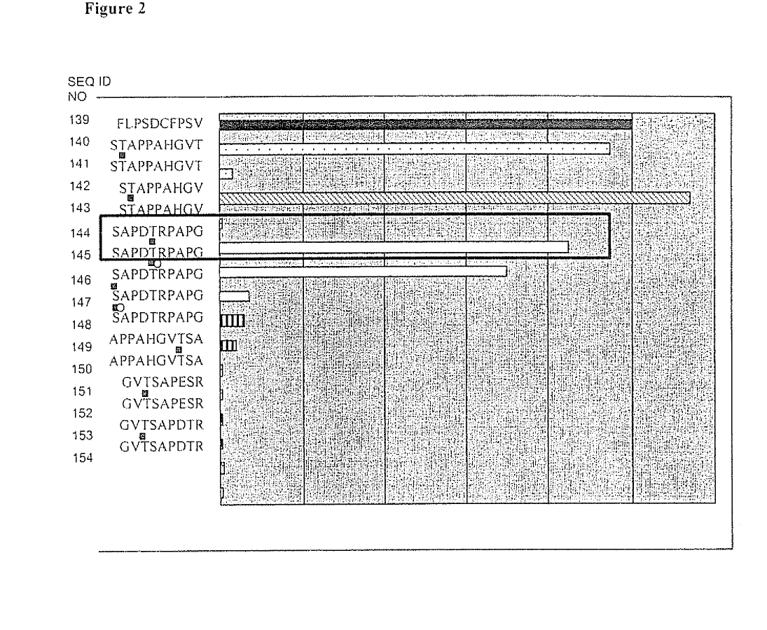

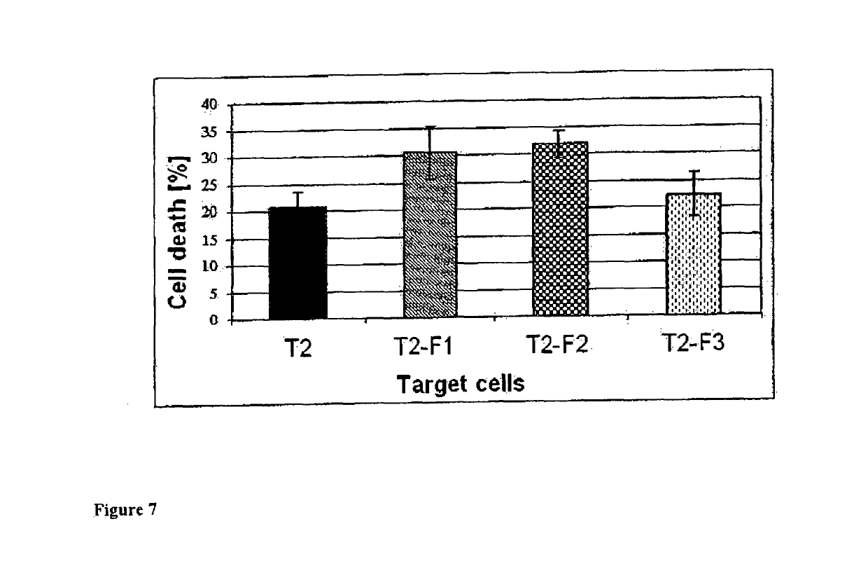

The MUC1 derived glycopeptides were chemically synthesized and kindly provided by Prof. Hans Paulsen (GP1-8, refer to table 1; SGGP1-5, refer to FIG. 1; F1-F3, refer to FIG. 7). The P1 (GVT100) and P2 (ESR61) peptides were provided by Dr. Olivera J. Finn and in vitro glycosylated with GalNAc using purified polypeptide GalNAc-transferases T1 and T2 (kindly provided by Dr. Henrik Clausen, School of Dentistry, University of Copenhagen, Denmark). Other non-glycosylated peptides were ordered from Mimotopes/Perbio and glycosylated in vitro. TAP25 was synthesized in a local facility at the Institute of Biochemistry, Cologne, Germany. For the in vitro glycosylation, peptide substrates (50 nmol) were dried from solutions in water by vacuum centrifugation and solubilized in 368 μl of a 25 mM cacodylate buffer containing 10 mM MnCI₂ (final pH 7.4). The addition of the co-substrate UDP-GalNAc (0.28 mg=112 μl of stock solution containing 2.5 mg/ml of cacodylate buffer) was followed by the respective enzyme preparations ppGalNAc-T1 and ppGalNAc-T2, 20 μl of ppGalNAc-T1 (2.0 mU/ml) and/or ppGalNAc-T2 (4.04 mU/ml) (18), to yield a total volume of 500 μl. The reaction mixtures were incubated for 48 h at 37° C. by adding fresh aliquots of co-substrate and enzyme(s) after a 24 h interval. The reaction was stopped by acidification with TFA to pH 2. The glycosylated peptide was purified from the reaction mixture by reverse-phase HPLC.

### In Vitro Proteolysis of Glycopeptides

A 1 mg/ml solution of immunoproteasomes prepared according to a published protocol (20) was purchased from Immatics Biotechnologies (Tübingen, Germany) and stored frozen at −80° C.

Glycopeptides were incubated with 10 μg of human 20S immunoproteasomes (Immatics) for 48 h, at 37° C., in 10 μl of digestion buffer (20 mM HEPES/KOH, pH 7.6 containing 2 mM Mg(CH3COO)2 and 1 mM DTT) as described (13). Digestion was stopped after 24 h by freezing the sample at −80° C. Human cathepsin-L was purchased from Sigma (Munich, Germany) and solubilized in 0.1 M sodium acetate, pH 5.5, containing 1 mM EDTA and 1 mM dithiothreitol (12). About 5 mU of enzyme were added to sialoglycopeptide substrates SGGP1 to SGGP5 (75-100 μM) in a total volume of 20 μl. The reaction mixtures were incubated at 37° C. for 24 h and 1 μl of trifluoroacetic acid was added to stop the reaction. The reaction products were cleaned by solid-phase extraction on ZipTipC18 pipette tips and 0.5 μl aliquots were applied onto the MALDI target for mass spectrometric analysis. Kinetic studies with the non-glycosylated 100-mer P1 were performed under the same conditions and the products were chromatographed on HPLC for purification and quantification.

### Purification of Glycopeptides by HPLC

Aliquots (50-100 μl) of the reaction mixtures were injected onto a narrow-bore ODS Ultrasphere column (150×2 mm; Beckman Instruments, Munich, Germany) for analytical chromatography or onto a Prevail C 18 column (Alltech, Munich, Germany) for preparative chromatography.

Chromatography was performed on an HPLC system (System Gold Beckman Instruments, Munich, Germany) by gradient elution in a mixture of Solvent A (H2O with 2% acetonitril and 0.1% trifluoroacetic acid, v/v) and Solvent B (80% acetonitril and 20% H2O with 0.1% trifluoroacetic acid, v/v). The gradient for analytical chromatography increased from 0% solvent B to 6% solvent B within 3 min, followed by an increase from 6% to 36% solvent B within 40 min. The gradient applied in preparative chromatography increased from 5% to 30% solvent B within 30 minutes. The glycopeptides were run at a flow rate of 0.3 ml/min on the narrow-bore ODS column and at a flow rate of 2 ml/min on a Prevail C18 column. The photometrical detection was performed at a wavelength of 214 nm.

### Structural Identification by Mass Spectrometry

Matrix-assisted laser desorption/ionization (MALDI)-MALDI analyses were performed on a Bruker-Reflex IV instrument (Brucker-Daltonics, Bremen, Germany) by positive ion detection in the reflectron mode.

Liquid chromatography (LC)-MS/MS data were acquired on a Q-TOF2 quadruple-TOF mass spectrometer (Micromass, Manchester, United Kingdom) equipped with a Z spray source.

### Cellular Processing and Presentation in a Mouse Dendritic Cell Model

Poly-lactic acid based vesicles were generated mixing two solutions: Solution A: 200 nmol of P2 peptide (1 mg) resolved in 3% cold water solution of poly-vinyl alcohol; Solution B: 5% poly-lactic acid in a mixture of ethanol and acetone (9:1, v/v). Solution B (30 ml) was slowly added to solution A (150 ml) under constant mixing and stirring overnight at room temperature. The next day the mixture was centrifuged at 10.000×g for 10 min and washed three times with PBS (200 ml), followed by centrifugation steps as mentioned previously. The generated vesicles were resuspended in 330 μl of PBS and added to media of 4×108 DC2.4 mouse dendritic cells growing confluent in nine cell culture flasks (300 cm²). The cells were grown as usual, but in exosome-depleted medium.

Cells were incubated with PLA-vesicles for 24 h at 37° C. and 5% CO2. The cell culture supernatant was collected and exosomes were isolated by ultracentrifugation (see below). Isolated exosomes were resuspended in 100 μl 0.1% TFA/H2O. Acidification of exosomes denatures tertiary MHC complex and releases bound peptide epitopes. The solution was shortly spun and injected on a HPLC narrow-bore ODS column for separation of eluted peptides and glycopeptides. Fractions were collected manually, dried and analyzed by MALDI-TOF-MS as described. Molecular masses of detected ions were analyzed by the FindPep software and by nanospray ESI-MS/MS analyses.

Exosomes were isolated from FCS or cell culture supernatant by subsequent centrifugation steps: 10 min/1200 g; 30 min/10000×g; 30 min/20.000×g; and 60 min/00.000×g. The exosomes were pelleted in the final centrifugation step, washed with PBS and used for further analyses. For the depletion of FCS from exosomes, exosomes from serum were pelleted by ultracentrifugation at 110.000×g for 1 h.

### MHC Stabilization Assay.

MHC stabilization assay is based on the increased affinity of HLA-A, -B, -C specific monoclonal W6/32 antibody for the stabilized conformation of MHC molecules after binding of a peptide ligand. The T2 cells express empty MHC molecules with short half-life.

For MHC stabilization assay T2 cells were grown in IMDM medium supplemented with 2% FCS. T2 cells were washed in PBS from the residual serum, and resuspended at 1×106/ml of IMDM supplemented with 0.1% FCS and 25 mM HEPES. Half of a million of cells were incubated in 48-well cell culture plate with 1 μg of □2-microglobulin and 100 μg of peptide for 16 h at 27° C. and 8% CO2. Cells were washed with 5 ml PBS, centrifuged at 180×g for 5 min and the pellet was resuspended in 10 μl PBS containing 1 μg of W6/32 antibody and incubated at 4° C. for 30 minutes.

Subsequently, cells were washed in PBS with 1% BSA (3×5 ml) and the pellet was resuspended in 50 μl of secondary anti-mouse Alexa 488 antibody (1:1000 in PBS with 0.1% BSA), incubated for 30 minutes in the dark, washed with PBS (3×5 ml) and resuspended in 500 μl of PBS. Binding of the W6/32 antibody to MHC class I molecules was analyzed by FACS on the Beckman Coulter ADCXL4C flow cytometer. As a positive control hepatitis B virus core antigen HBVc18-27 P6Y˜C (FLPSDCFPSV) was used. As a negative control T2 cells were used, to which no peptide was added.

Relative binding capacities of the peptides were expressed as the percent of the positive control signal (100%), normalized to the negative control (0%). A mean fluorescence that was at least 10% higher than the negative control level was considered positive binding.

### In Vitro Priming of Peptide-Specific CTL

Peripheral blood mononuclear cells (PBMCs) were isolated from buffy coats of HLA-A*0201 healthy donors by Ficoll-Hypaque (MP Biomedicals, Solon, Ohio) density gradient centrifugation.

Dendritic cells (DCs) were generated by culturing adherent monocytes for 6 days in AIM-V medium (Invitrogen, Carlsbad, Calif.) supplemented with 500 U/mL granulocyte macrophage colonystimulating factor (GM-CSF, R&D, Minneapolis, Minn.) and 1000 U/mL interleukin (IL)-4 (R&D), and then matured for 48 hours with a cocktail of cytokines consisting of 10 ng/mL of each, tumor necrosis factor (TNF)-alpha, IL-6, IL-1beta (R&D), and 1 μg/mL prostaglandine E2 (Sigma, St. Louis, Mo.). Maturity and activation of DCs was confirmed by flow cytometry (FACS LSRII, BD) 9 using monoclonal antibodies (BD, San Diego, Calif.) against CD80, CD14 and CD11 c. Matured DCs were pulsed for 4 hours in RPMI 1640 medium (Lonza BioWhittaker, Walkersville, Md.) supplemented with 10% human AB serum (GemCell, Gemini BioProducts, Woodland, Calif.), 2 mM L-glutamine (Mediatech cellgro, Herndon, Va., USA), 100 units/ml penicillin and 100 μg/ml streptomycin (Mediatech cellgro), and 1% nonessential amino acids (Gibco-Invitrogen), with the synthetic MUC1-derived peptides F1, F2 or F3 at the concentration of 30-50 μg/106 DC. DCs pulsed with single peptides were combined, washed, resuspended and used as stimulator cells. They were added to the autologous lymphocyte fraction in 24-well Linbro plates (ICN, Aurora, Ohio) at a ratio of 1:10 at culture initiation and fed in RPMI 1640 media supplemented with 10% human AB serum, 10 ng/ml rhIL-6 (R&D) and 5 ng/ml rhIL-12 (PreproTech, Rocky Hill, N.J.). On day 10 and thereafter every 9 days, T cells were restimulated at a ratio of 1 DC to 20 T cells, in RPMI 1640 media supplemented with 10% human AB serum, rhIL-2 and rhIL-7 (20 U/ml each, R&D).

### T-Cell Assays

Antigen Specificity was Assessed after 4 Stimulations. Three Days Past Each stimulation cell supernatant was collected and IFNy secretion was quantified using OptElA human IFNy ELISA kit (BD). CD8+ T-cells were enriched using magnetic microbeads (human CD8+ T Cell Isolation Kit II from Miltenyi Biotech, Auburn, Calif.) according to the manufacture's instructions and analyzed by flow cytometry for intracellular IFN-gamma and TNF-alpha production after a final 5-hour restimulation with peptide-loaded DCs in the presence of monensin (Golgi Stop, BD; 21). CTL activity was determined 3 days after the last restimulation. T2 target cells were incubated over night at 37° C. in the presence of F1 [SAPDTRPAPG], F2 [SAPDT(GalNAc)RPAPG] or F3 peptides [SAPES(GalNAc)RPAG] at 10 μg/ml concentration each. They were washed, labeled with 5- (and 6)-carboxyfluorescein diacetate succinimidyl ester (CFSE) according to the manufacture's instructions (Immunochemistry, Bloomington, Minn.) and added to a 96-well plate in duplicates at 5000 cells/well. CFSE labeled T2 cells not loaded with peptides were used as controls. CD8+ T effector cells were added at various E:T ratios for a 6-hour incubation at 37° C. Target cell death was detected by adding 7-amino-actinomycin (7-AAD, Immunochemistry) to each well 10 min before data analysis by flow cytometry and analysis was performed according to the manufacture's instructions (22).

### Molecular Modelling.

Molecular modelling was performed by the SYBYL 7.1 software package (Tripos, Inc., St. Louis, Mo., USA). Two crystal structures of the HLA-A2/10-mer peptide complexes (PDB IDs: 1HHH and 1l4F) were used as templates for modelling of HLA-A2/SAPDTRPARG (HLA/SAP10), either non-glycosylated or glycosylated at Thr5 with GalNAc (T5*) and Gal-GalNAc (T5**) residues.

Template peptides were converted to SAP10 sequence and energy-minimized within the binding groove of the HLA-A0201 molecule. Two orientations of the sugar moiety, pointed into the groove and out of the groove, were considered for the glycosylated Thr5. The molecular structures were energy-minimized in a vacuum by using the conjugate gradient algorithm (the Powell method implemented in SYBYL) to a maximum derivative of 0.05 kcal/(mol*Å). Tripos force-field with Kollman-all-atom partial charges for the MHC molecule and Gasteiger-Marcili charges for(glyco)peptide was used in structure optimization and in subsequent FlexiDock runs. The distancedependent dielectric of 4r was used to mimic aqueous solvent environment. The energy-minimized structures were analyzed for atomic fluctuations and hydrogen bonding pattern and were used for docking with the FlexiDock module of SYBYL. All single bonds of the (glyco)peptide ligand were considered as rotatable in FlexiDock runs, except amide and ring bonds. All figures were made with SYBYL.

### RESULTS

Glycopeptides with Potential MHC Class I Binding Capacity are Formed by Immunoproteasomal Cleavage of Muc1-Derived Substrates

The processing of O-glycosylated MUC1 within cross-presentation pathways and MHC class I presentation is likely to occur by immunoproteasomes. We could address whether immunoproteasomes can generate fragments of the O-glycosylated MUC1 tandem repeat domain that would fit into the MHC class I groove and whether glycosylation introduces specific restrictions to the process.

The in vitro digestion of non-glycosylated substrates followed by chromatography and mass spectrometry and analyses of the derived fragments revealed that:1) longer substrates (100-mer and 61-mer) in average yielded fragments of 20 amino acids with about 20% of peptides that ranged in sizes fitting to the MHC class I groove (8- to 11-mers); and 2) shorter, substrates (20-mers) yielded in average 11-meric fragments and higher amounts of 8- to 11-meric peptides (50%) (Table 1). O-Glycosylation of specific positions in the sequence did not change the average fragment length, except in the case of glycosylation at the central Thr/Ser within DTR/ESR, which reduced the fraction of 8- to 11-mers to 15/35% (GP3 and GP6), respectively (Table 2). The 8- to 11-mers were mainly formed by two cleavages within the preferred processing regions: SAP and GST (Table 3). Consequently, the glycosylation position significantly influenced the processing and therefore the amount of glycopeptides within the group of 8- to 11-mers. Thr5 is located in the N-terminal of the main processing region (SAP) and is found in only 18% of the generated fragments.

The DTR/ESR glycosylated GP3 and GP6 yielded exclusively glycosylated 8- to 11-meric epitopes (98%). The GST glycosylated GP4 and GP7 released considerable amounts of glycosylated 8- to 11-mers (40%).

In summary, shortening of the substrate from 100-meric to 20-meric peptides significantly increased the generation of 8-11-meric processing products (potential MHC class I ligands). The introduction of GalNAc specifically directed the formation of glycosylated 8- to 11-mers. The DTR/ESR glycosylated peptides, although releasing lower overall levels of 8- to 11-mers compared to other glycopeptides, generated these almost exclusively in a glycosylated form, and were the best source of glycopeptides potentially binding to MHC.

Cathepsin-L is Able to Generate Peptides and Glycopeptides In Vitro that Resemble the Patterns of Immunoproteasomal Cleavage

Further we could show whether cathepsin L is able to cleave complex glycosylated MUC1 repeats into fragments that fit to the size limitations of MHC class I binding. The in vitro digestion of non-glycosylated 100-mer and chromatography of the reaction products by HPLC revealed 1) quantitative digestion of the substrate within 24 h; 2) formation of a complex pattern of fragments ranging in size from 7-mers to 33-mers (FIG. 1A) that compares to a more restricted pattern in the cellular processing; 3) three major and one minor cleavage sites within the repeat peptide; and 4) the STA10 fragment as one of the dominant products. Similar results were obtained with the non-glycosylated 61-mer comprised of three MUC1 repeats with variant sequences (DT>ES).

To find out whether peptides glycosylated at tolerated sites (DTR or GST) with sialyltrisaccharides (as found on cancer-associated mucin), would form effective substrates and yield reaction products in the appropriate size range for MHC class I binding.

A series of sialoglycopeptides was tested for this purpose (see Table 2), which have a glycosylation with the trisaccharide NeuAc2-3 Gall-3GalNAc in common. As previously shown for glycopeptides with GalNAc substitution, the sialoglycopeptides with glycosylation at Thr or Ser in the VTSA motif (SGGP1 to SGGP3 and SGGP5) were not cleaved within this motif, whereas the tri-glycosylated species SGGP4 lacking glycan substitution at these two positions was an effective substrate. SGGP1 and SGGP2 instead showed cleavage of the Gly-Ser bond in the GSTA motif. In SGGP4 the Gly-Ser linkage in the GST motif was resistant to cleavage, probably because of a sterical hindrance by the adjacent glycans. However, a new cleavage site was used by the enzyme in SGGP4: the Thr-Arg linkage in the DTR motif (see SGGP4 in FIG. 1B). The major product of SGGP4 digestion was accordingly the RPA11 glycopeptide, a fragment with potential MHC class I binding capacity.

In summary, we could reveal evidence for the formation of peptides and glycopeptides by in vitro proteolysis of MUC1 repeats with cathepsin L that fall into the size range of class I binding peptides. The O-linked glycans strongly affect proteolytic cleavage in a site specific manner.

### Mouse Dendritic Cells Simulate Cleavage Patterns of Human Immunoproteasomes and Cathepsin-L

To exclude in vitro artefacts the processing of the MUC1 repeat sequence on the cellular level was analyzed.

Cross-presentation of MUC1 61-mer was performed in the mouse dendritic cell line DC2.4. The loading of poly-lactic vesicles with antigen resulted in the generation of high amounts of MUC1-derived MHC epitopes that could be identified on the peptidomic level by mass spectrometry. After incubation of mouse dendritic cells DC2.4 with poly-lactic acid vesicles (PLA) carrying P2 peptide, exosomes produced by the cells were collected by ultracentrifugation, acidified and fractionated by reverse phase HPLC. Collected fractions were dried by vacuum centrifugation and analyzed by MALDI/MS. The molecular masses of detected ions were analyzed by the FindPep software application. The mass spectra revealed the presence of at least 18 distinct peptide species in the size range from 8- to 25-mers (Table 4). Eight peptides were in the class I size range (8- to 11-mers), while ten fell into the class II size range (12- to 25-mers). Among the peptides with potential class I binding activity the major species were identical to those found in the cell-free in vitro studies (APE8, APE9, APE10, STAL 10, SAP 10, SAP11, PES9, and PES 10). The fact that these peptides were eluted from DC-derived exosomes indicates considerable class I binding activity. Their structural identity with fragments generated by immunoproteasomes in vitro clearly validates the results of cell-free processing studies.

### Prediction of Peptide Binding to MHC Class I Allele HLA-A2

In the digests of all analyzed 21-meric glycopeptides, 117 fragments in the size range of 8 to 11 amino acid long peptides and glycopeptides were generated. To identify MHC binding glycopeptides, we performed a pre-selection based on the predicted ligation strength of the peptide. The ligation strength of non-glycosylated 8- to 11-meric sequences to MHC class I was analyzed by the SYFPEITHI MHC-epitopes predicting software (http://www.syfpeithi.de/). Only 52 amino acid sequences were analyzed, because the variation due to glycosylation had to be ignored (the influence of glycans on MHC binding is unknown) as well as 11-meric sequences. Analyses concentrated on the HLA-A0201, as the most frequent MHC class I allele (Table 5). Peptides predicted as good binders were glycosylated and experimentally tested by MHC stabilization assay.

### Glycans Modulate Binding of Peptides to HLA-A2

The predicted good binders with a potential glycosylation site were synthesized, glycosylated in vitro and tested for binding to empty HLA-A0201 molecules of T2 cells by MHC stabilization assay. Ligation strength measured by flow cytometry was compared to the positive control: FLPSDCFPSV peptide and to non-loaded T2 cells as the negative control. Results for glycosylated peptides were compared with affinities of their non-glycosylated forms. FIG. 2 presents the ligation strengths of tested peptides compared with a negative control considered as 0% and positive control considered as 100%. As predicted by the SYFPEITHI software tool, the strongest binders in the MHC stabilization assays were STA9, STA10 and SAP10. A strong reduction of ligation strength was caused by the introduction of GalNAc into STA9 and STA10, reducing it to the level of the negative control. An exception represents the glycopeptide SAPDT(-GalNAc)RPAPG (SAP10-T5*), which was revealed as the only MUC1 VNTR glycopeptide binding comparably strong in the glycosylated and non-glycosylated form. All other peptides lost their binding abilities after glycosylation or were already inactive in the non-glycosylated form.

### Modelling of Glycopeptide Binding

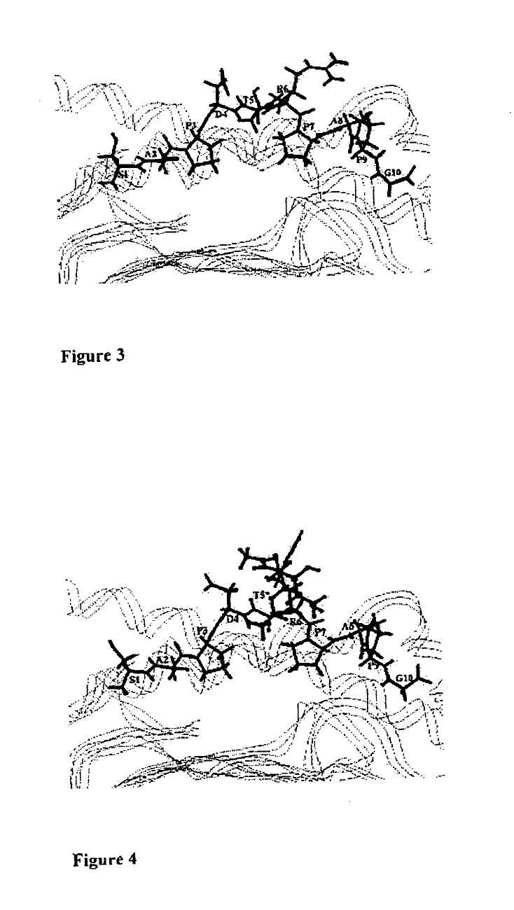

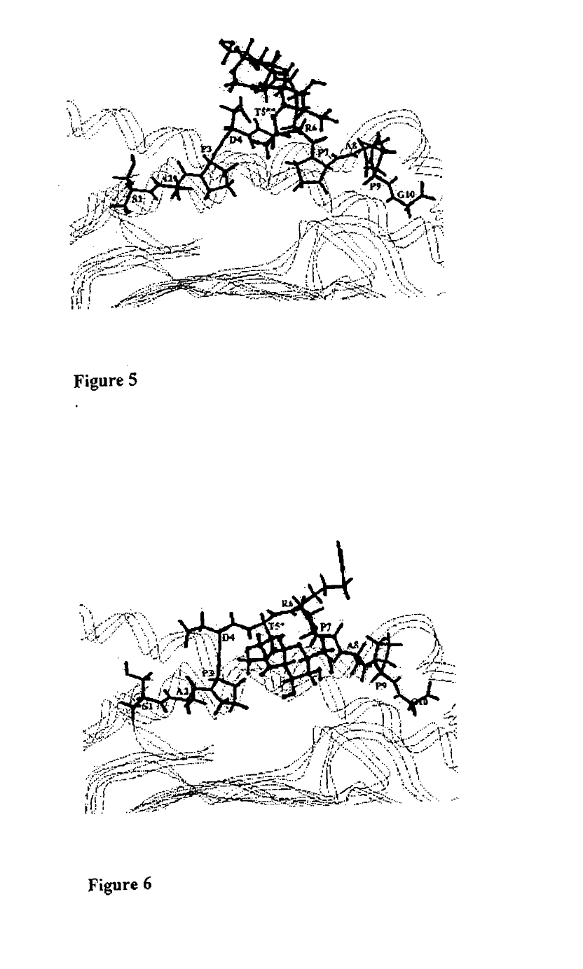

Each model of the MHC/(glyco)peptide complex included a 10-residue (glyco)peptide and 180 residues of the MHC heavy chain that formed the binding groove. The groove lies between the long a1 and a2 helices and the b-sheet platform. For all models, the peptides were mostly buried into the MHC groove and demonstrated extended conformations with slightly bulged central parts P4-P6 (FIGS. 3-6). The modelled complexes maintained very similar patterns of specific interactions between the side-chain atoms of MHC molecule and the main-chain atoms of peptide near N- and C-termini that resulted in a conserved set of hydrogen bonds. All models demonstrated high similarity to the template crystal structures with root-mean-square deviations of the modelled atomic coordinates from the corresponding template that were less than 0.15 Å for the heavy atoms of the peptide backbone. The crystal structure of the HLA-A2/FLPSDFFPSV complex (1 HHH) was used as a template to model the reference structure for the comparison of binding energies calculated with FlexiDock. The 10-residue peptide FLPSDFFPSV is similar to the non-glycosylated peptide FLPSDCFPSV that was used as a positive control in the MHC stabilization assays. After converting F6 to C6, the HLA/FLPSDCFPSV complex was energy-minimized and used as a starting structure for molecular docking calculations with FlexiDock. No significant atomic fluctuations from the crystal structure or hydrogen-bonding changes were detected. The FlexiDock run resulted in eight docked structures of the HLA/FLPSDCFPSV complex with the lowest binding energy equal to −241 kcal/mol. All docked peptide backbone conformations closely resembled the crystal structure with slight variations of side-chain conformations. From four known crystal structures of the HLA-A2/10-mer peptide complexes, only two (1 HHH and 1l4F) were converted to the SAP10 sequence with an attached sugar moiety without significant structural perturbations of the peptide backbone. To model the HLA/SAP10 complex from the 1 HHH-based template, the FLPSDFFPSV sequence was converted to the non-glycosylated SAPDTRPARG and energy-minimized. The FlexiDock run generated 10 docked structures with the best binding energy equal to −226 kcal/mol. The generated peptide backbone conformations demonstrated very similar structural features with S1, A2, and P3 residues positioned at the binding pockets A, B, and D; and with P7 and G10 positioned at the E and F binding pockets, respectively. The non-glycosylated T5 residue was positioned at the pocket C; whereas D4 and R6 were mostly exposed out of the MHC binding groove (FIG. 3). The significant conformational variations were only observed for the side-chain conformations of the D4 and R6 residues of the docked peptide. To model binding of glycosylated peptides to MHC, the T5 residue of the HLA/SAP10 complex was converted to Thr-GalNac (T5*) and Thr-Gal-GalNAc (T5**) with torsion angles corresponding to the lowest energy conformation of the glycosylated threonine. After energy minimizations and subsequent FlexiDock runs, orientations of the sugar moieties for both T5* and T5** were found to be directed out of the MHC groove for all low-energy conformations (FIGS. 4 and 5). The side-chain orientations directed into the groove were prohibited due to significant intermolecular clashes of the sugar moieties with the amino acid residues A69, H70, T73, and H74 of the C pocket of the HLA-A0201 molecule. The lowest binding energies for the HLA/SAP10 complexes glycosylated at T5* and T5** were equal to −216 kcal/mol and −208 kcal/mol, correspondingly. To explore the possibility of different sugar orientations, the HLA/GVYDGREHTV complex (114F) with native D4 and R6 residues at corresponding positions was converted to the SAPDTRPARG sequence and energy-minimized. This template allowed us to model the HLA/SAP10 complex glycosylated at T5* with GalNAc to be directed either into or out of the MHC groove. The lowest binding energy for the HLA/SAP10 complex without sugar was equal to −238 kcal/mol. For the glycosylated T5* oriented into the groove (FIG. 6), the binding energy was equal to −244 kcal/mol; whereas, for T5* oriented out of the groove, the lowest binding energy was equal to −221 kcal/mol. For the HLA/SAP10(T5**) complex with the larger Gal-GalNAc residue at T5**, the allowed side-chain orientations of the T5** were only directed out of the MHC groove. The best binding energy for the SAP10(T5**) was equal to −213 kcal/mol. Thus, our docking experiments suggested a better binding for the SAP10(T5*), if the sugar moiety was oriented into the HLA binding groove; whereas for the GalNAc and Gal-GalNAc residues directed out of the groove, the predicted binding was significantly weaker. It should be noted that the binding energies calculated from the docking experiments using the FlexiDock module of SYBYL provide rather qualitative estimates of the (glyco)peptide binding that could serve as the relative scoring binding propensities of the potential (glyco)peptide epitopes.

### Priming of Glycopeptide-Specific T-Cells

Lymphocytes from a HLA-A*0201 healthy donor were repeatedly stimulated in vitro with autologous DCs pulsed with F1 [SAPDTRPAPG], F2 [SAPDT(GalNAc)RPAPG] and F3 [SAPES(GalNAc)RPAPG] for a period of 4 weeks. CD8+T-cells were then enriched by negative selection using magnetic microbeads and specific immunoreactivity of the induced CTLs was tested with T2 target cells loaded with the relevant peptides or no peptides. Stimulated T-cells secreted large amounts of IFN-γ, but not TNF-a into the cell supernatant during the 4-week culture period. A peak in IFN-γsecretion (>23 ng/ml) was reached after the first restimulation with autologous peptide-loaded DCs (not shown). After the last cycle of restimulation (4 weeks of in vitro culture) 28.8% of F1-stimulated CD8+ cells, 22% of F2-induced CD8+T-cells and 18.1% of F3-induced CD8+T-cells produced IFN-γ, but not TNF-α(not shown). Three days after the last restimulation the induced CD8+ T-cells secreted >17 ng/ml IFN-γ, indicating an immunological response and activity. The responder cells were tested for cytotoxic activity and as shown in FIG. 7, CD8+ T cells efficiently lysed T2 target cells pulsed with F1 or F2 peptide, whereas T2 cells loaded with F3 peptide showed only minimal increased cell lysis above the background lysis of unpulsed T2 cells. These results demonstrate that the induced CTL recognized F1 and F2 antigen-peptides specifically and were able to target cells presenting those peptides.

TABLE 1
Number of fragments generated by immunoproteasomes and average
fragment length in the digest of MUC1 repeat peptides and glycopeptides.
REPLACEMENT SHEET

| | | | NUMBER OF | AVERAGE FRAGMENT |
|---|---|---|---|---|
| NAME | SEQ ID No. | SEQUENCE | FRAGMENTS | LENGTH |
| P1 | 13 | (CVISAPDTRPAPGSTAPPAH)x5 | 105 | 25.2 |
| P2 | 14 | A(HGVTSAPESRPAPGSTAPPA)x3 | 105 | 17.5 |
| P3 | 15 | AHGVTSAPDTRPAPGSTAPPA | 29 | 10.8 |
| P4 | 16 | AHGVTSAPESRPAPGSTAPPA | 31 | 11.6 |
| GP2 | 17 | HGVTSAPDTRPAPGSTAPPA | 63 | 11.7 |
| GP3 | 18 | AHGVTSAPDTRPAPGSTAPPA | 33 | 10.9 |
| GP4 | 19 | AHGVTSAPDTRPAPCSTAPPA | 47 | 12.9 |
| GP5 | 20 | AHGVTSAPESRPAPGSTAPPA | 46 | 11.6 |
| GP6 | 21 | AHGVTSAPESRPAPGSTAPPA | 46 | 10.6 |
| GP7 | 22 | HGVTSAPESRPAPGSTAPPA | 61 | 11.7 |
| GP8 | 23 | AHGVTSAPESRPAPGSTAPPA | 46 | 10.5 |

Non-glycosylated and glycosylated peptides with AHG starting motif were processed by immunoproteasomes, and analyzed by reverse phase HPLC and MALDI mass spectrometry followed by identification and quantification of fragments (13).

TABLE 2
Relative amounts of 8- to 11-meric (A) and glycosylated proteolytic
fragments (B).

| | PEPTIDE | P1 | P2 | P3 | P4 | GP2 | GP3 | GP4 | GP5 | GP6 | GP7 | GP8 |
|---|---|---|---|---|---|---|---|---|---|---|---|---|
| A | 8-11 mers | 13 | 26 | 52 | 50 | 55 | 15 | 48 | 79 | 35 | 29 | 35 |
| | (%) | | | | | | | | | | | |
| B | % of | 0 | 0 | 0 | 0 | 18 | 98 | 45 | 26 | 96 | 39 | 21 |
| | glycopeptide | | | | | | | | | | | |

Relative amounts of potentially MHC class I fitting octa- to undecapeptides were calculated and expressed relative to the total amount of digestion products generated by immunoproteasomal cleavage of the respective substrate (A). The percentage of glycosylated 8- to 11-meric fragments was calculated relative to the fraction of total 8- to 11-mers (B).

TABLE 3
Relative frequencies of 8- to 11-meric proteolytic fragments grouped
according to their N-terminus.

| | AHG 8 to -11 | SAP8 to -11 | PAP8 to -11 |
|---|---|---|---|
| GP2 | 18% | 42% | 40% |
| GP3 | 20% | 78% | 2% |
| GP4 | 3% | 60% | 37% |
| GP5 | 26% | 27% | 47% |
| GP6 | 55% | 42% | 3% |
| GP7 | 8% | 61% | 31% |

The 8- to 11-meric fragments in digests of MUC1 repeat glycopeptides were grouped according to their N-terminal tripeptide motif into three groups and the relative amounts in each group were calculated (expressed as % of the total amount of 8- to -11-mers). Dark squares mark the position of glycosylated threonine within the VTS (GP2, GP5), DTR/ESR (GP3/GP6) or STA motifs (GP4/GP7).

TABLE 4

REPLACEMENT SHEET
Peptides eluted from exosomal MHC molecules after cross-presentation of
MUC1 100-mer in a murine dendritic cell line

| | | | |
|---|---|---|---|
| MW(Da) | Potential MHC class I | MW(Da) | Potential MHC class II binding |
| (m/z) | binding 8- to 11-mers | (m/z) | 12- to 25-mers |

| | | | | | |
|---|---|---|---|---|---|
| 823 | SEQ ID NO. 24 | APESRPAP | 1169 | SEQ ID NO. 34 | GVTSAPESRPAP |
| | | | | SEQ ID NO 35 | VTSAPESRPAPG |
| 844 | SEQ ID NO. 25 | TSAPESRP | 1264 | SEQ ID NO. 36 | PESRPAPGSTAPP |
| 881 | SEQ ID NO. 26 | APESRPAPG | 1523 | SEQ ID NO. 37 | TSAPESRPAPGSTAPP |
| 896 | SEQ ID NO. 27 | PESRPAPGS | 1549 | SEQ ID NO. 38 | HGVTSAPESRPAPGST |
| 937 | SEQ ID NO. 28 | STAPPAHGVT | 1629 | SEQ ID NO. 39 | SAPESRPAPGSTAPPAH |
| | SEQ ID NO. 29 | TAPPAHGVTS | | | |
| 968 | SEQ ID NO 30 | SAPESRPAPG | 1692 | SEQ ID NO 40 | AHGVTSAPESRPAPGSTA |
| | SEQ ID NO 31 | APESRPAPGS | | SEQ ID NO 41 | VTSAPESRPAGSTAPPA |
| 999 | SEQ ID NO. 32 | PESRPAPGST | 1786 | SEQ ID NO. 42 | SAPESRPAPGSTAPPAHGV |
| 1056 | SEQ ID NO. 33 | SAPESRPAPGS | 1986 | SEQ ID NO. 43 | VTSAPDTRPAPGSTAPPAHGV |
| | | | 2323 | SEQ ID NO. 44 | TAPPAHGVTSAPESRPAPGSTAPPA |
| | | | 2459 | SEQ ID NO. 45 | TSAPESRPAPGSTAPPAHGTSAPES |

TABLE 5
HLA-A*0201 allele binding prediction of proteasomal products.

| | | LIGATION |
|---|---|---|
| SEQ ID NO. | FRAGMENT | STRENGTH |
| 46 | SAPESRPAP | 10 |
| 47 | SAPESRPAPG | 10 |
| 48 | TSAPDTRPA | 10 |
| 49 | PGSTAPPA | 9 |
| 50 | RPAPGSTA | 9 |
| 51 | SAPDTRPA | 9 |
| 52 | SAPDTRPAP | 9 |
| 53 | SAPDTRPAPG | 9 |
| 54 | SAPESRPA | 9 |
| 55 | SRPAPGSTA | 9 |
| 56 | VTSAPESRPA | 9 |
| 57 | PAPGSTAPPA | 8 |
| 58 | TSAPESRPA | 8 |
| 59 | APGSTAPPA | 7 |
| 60 | PAPGSTAPP | 7 |
| 61 | RPAPGSTAP | 7 |
| 62 | TRPAPGSTA | 7 |
| 63 | GVTSAPDTR | 6 |
| 64 | GVTSAPESR | 6 |
| 65 | RPAPGSTAPP | 6 |
| 66 | AHGVTSAPES | 5 |
| 67 | ESRPAPGST | 5 |
| 68 | ESRPAPGSTA | 5 |
| 69 | AHGVTSAPE | 4 |
| 70 | APDTRPAPG | 4 |
| 71 | GVTSAPDTRP | 4 |
| 72 | GVTSAPESRP | 4 |
| 73 | APESRPAPG | 3 |
| 74 | APESRPAPGS | 3 |
| 75 | HGVTSAPES | 3 |
| 76 | PESRPAPGST | 3 |
| 77 | SRPAPGSTAP | 3 |
| 78 | HGVTSAPDTR | 2 |
| 79 | HGVTSAPESR | 2 |
| 80 | PDTRPAPGST | 2 |
| 81 | TRPAPGSTAP | 1 |
| 82 | GVTSAPDT | 0 |
| 83 | GVTSAPES | 0 |
| 84 | HGVTSAPD | 0 |
| 85 | PAPGSTAP | 0 |
| 86 | PDTRPAPG | 0 |
| 87 | PDTRPAPGS | 0 |
| 88 | PESRPAPG | 0 |
| 89 | PESRPAPGS | 0 |
| 90 | SRPAPGST | 0 |
| 91 | TRPAPGST | 0 |
| 92 | TSAPDTRP | 0 |
| 93 | TSAPESRP | 0 |
| 94 | VTSAPDTR | 0 |
| 95 | APGSTAPP | 0 |
| 96 | AHGVTSAPDTR | 0? |
| 97 | AHGVTSAPESR | 0? |
| 98 | APDTRPAP | 0? |
| 99 | APDTRPAPGST | 0? |
| 100 | APESRPAP | 0? |
| 101 | APESRPAPGST | 0? |
| 102 | DTRPAPGS | 0? |
| 103 | DTRPAPGSTAP | 0? |
| 104 | ESRPAPGS | 0? |
| 105 | ESRPAPGSTAP | 0? |
| 106 | HGVTSAPESRP | 0? |
| 107 | PDTRPAPGSTA | 0? |
| 108 | PESRPAPGSTA | 0? |
| 109 | RPAPGSTAPPA | 0? |
| 110 | SAPDTRPAPGS | 0? |
| 111 | SAPESRPAPGS | 0? |
| 112 | SRPAPGSTAPP | 0? |
| 113 | TRPAPGSTAPP | 0? |
| 114 | TSAPDTRPAPG | 0? |
| 115 | TSAPESRPAPG | 0? |
| 116 | VTSAPDTRPAP | 0? |
| 117 | VTSAPESRPAP | 0? |
| 118 | VTSAPESR | 0 |

Altogether 52 identidied 8- to 10-metric fragments from in vitro processing were analyzed by the SYFPEITHI software tool for their ligation strength to the human HLA-A*0201 allele. The information about glycosylation position was not included in these analyses due to software restrictions.

1. Tumour vaccines, especially for activation of glycopeptide-specific cytotoxic t-cells by MHC class I pathway, comprising at least one peptide of 8-11 amino acids derived from the region SAPDTRPAPGST of the human epithelial mucin MUC1 containing the immunodominant PDTRPAP region and which is glycosylated on threonine of the immunodominant PDTRPAP region and start with SAP, APD or PDT at it's N-terminus.

2. Tumor vaccines of claim 1, comprising at least one peptide with a length of 9-10 amino acids.

3. Tumor vaccines of claim 1, comprising at least one of the peptides
SAPDT(-GalNAc)RPAPG;
SAPDT(-Galβ-1, 3-GalNAc)RPAPG;
SAPDT(-GalNAc)RPAP;
SAPDT(-Galβ-1, 3-GalNAc)RPAP;
APDT(-GalNAc)RPAPG;
APDT(-Galβ-1, 3-GalNAc)RPAPG;
APDT(-GalNAc)RPAPGS;
APDT(-Galβ-1, 3-GalNAc)RPAPGS;
PDT(-GalNAc)RPAPGS;
PDT(-Galβ-1, 3-GalNAc)RPAPGS;
PDT(-GalNAc)RPAPGST;
PDT(-Galβ-1, 3-GalNAc)RPAPGST.

4. Tumor vaccines of claim 1, comprising the peptide SAPDT(-GalNAc)RPAPG.

5. Tumor vaccines of claim 1, comprising the peptide SAPDT(-GalNAc)RPAP.

6. Tumor vaccines of claim 1, comprising at least one of the peptides
APDT(-GalNAc)RPAPG;
APDT(-GalNAc)RPAPGS;
PDT(-GalNAc)RPAPGS;
PDT(-GalNAc)RPAPGST.

7. Tumor vaccines of claim 1, comprising at least one of the peptides
APDT(-Galβ3-1, 3-GalNAc)RPAPG;
APDT(-Galβ-1, 3-GalNAc)RPAPGS;
PDT(-Galβ-1, 3-GalNAc)RPAPGS;
PDT(-Galβ-1, 3-GalNAc)RPAPGST.

8. Tumor vaccines of claim 1, wherein the threonine is O-glycosylated.

9. Tumor vaccines of claim 1, wherein the glycosylation of the threonine is a monosaccharide.

10. Tumor vaccines of claim 1, wherein the glycosylation of the threonine is an α-acetylgalactosamine (GalNAc).

11. Tumor vaccines of claim 1, wherein the glycosylation of the threonine is a disaccharide.

12. Tumor vaccines of claim 1, wherein the glycosylation of the threonine is a Galβ-1, 3-GalNAc.

13. Tumour vaccines of claim 1, comprising at least one peptide of 8-11 amino acids derived from the region SAPDTRPAPGST of the human epithelial mucin MUC1 containing the immunodominant PDTRPAP region and which is glycosylated on threonine of the immunodominant PDTRPAP region and start with APD or PDT at it's N-terminus.

14. The synthetic peptides
SAPDT(-GalNAc)RPAPG;
SAPDT(-Galβ3-1, 3-GalNAc)RPAPG;
SAPDT(-GalNAc)RPAP;
SAPDT(-Galβ-1, 3-GalNAc)RPAP
APDT(-GalNAc)RPAPG;
APDT(-Galβ-1, 3-GalNAc)RPAPG;
APDT(-GalNAc)RPAPGS;
APDT(-Galβ-1, 3-GalNAc)RPAPGS;
PDT(-GalNAc)RPAPGS;
PDT(-Galβ-1, 3-GalNAc)RPAPGS;
PDT(-GalNAc)RPAPGST;
PDT(-Galβ-1, 3-GalNAc)RPAPGST.

15. A synthetic peptide according to claim 14, wherein the peptide is SAPDT(-GalNAc)RPAPG.

16. A synthetic peptide according to claim 14, wherein the peptide is SAPDT(-GalNAc)RPAP.

17. The synthetic peptides according to claim 14, wherein the peptides are
SAPDT(-GalNAc)RPAPG;
SAPDT(-Galβ-1, 3-GalNAc)RPAPG;
SAPDT(-GalNAc)RPAP;
SAPDT(-Galβ1, 3-GalNAc)RPAP.

18. The synthetic peptides according to claim 14, wherein the peptides are APDT(-GalNAc)RPAPG;
APDT(-Galβ-1, 3-GalNAc)RPAPG;
APDT(-GalNAc)RPAPGS;
APDT(-Galβ-1, 3-GalNAc)RPAPGS;
PDT(-GalNAc)RPAPGS;
PDT(-Galβ1, 3-GalNAc)RPAPGS;
PDT(-GalNAc)RPAPGST;
PDT(-Galβ-1, 3-GalNAc)RPAPGST.

19. A method of producing a peptide of claim 1 comprising the steps
a) providing a peptide comprising a tandem repeat domain of MUC1 or a part thereof, which at least contains one repeating unit of said tandem repeat domain of MUC1 and a glycosylation with a monosaccharide or a disaccharide at the threonine of the immunodominant PDTRPAP region;
b) contacting the peptide of a) with an effective amount of human immunoproteasomes or cathepsin L or a closely related enzyme hereof, thereby cleaving the peptide; and
c) isolating the fragments produced in b).

20. A method of producing a peptide of claim 1, wherein in vitro proteolysis of MUC1 repeats with cathepsin L of 2-100 meric peptides, preferably 11-30 meric peptides is used.

21. Antigen presenting cells (APC's) pulsed with antigens from claim 1, which are capable of inducing effective immune responses by activation of glycopeptide-specific cytotoxic T-cells through MHC class I pathways.

22. A therapeutic composition comprising a therapeutical effective amount of glycopeptides of claim 1 and optionally a pharmaceutically acceptable carrier.

23. A therapeutic composition comprising a therapeutical effective amount of APC's of claim 17 and optionally a pharmaceutically acceptable carrier.


SEQUENCE LISTING (154 sequences)

SEQ ID NO: 1 - protein, 10 aa, Artificial
  synthetic and modified peptide derived from human MUC1
SAPDTRPAPG

SEQ ID NO: 2 - protein, 10 aa, Artificial
  synthetic and modified peptide derived from human MUC1
SAPDTRPAPG

SEQ ID NO: 3 - protein, 9 aa, Artificial
  synthetic and modified peptide derived from human MUC1
SAPDTRPAP

SEQ ID NO: 4 - protein, 9 aa, Artificial
  synthetic and modified peptide derived from human MUC1
SAPDTRPAP

SEQ ID NO: 5 - protein, 9 aa, Artificial
  synthetic and modified peptide derived from human MUC1
APDTRPAPG

SEQ ID NO: 6 - protein, 9 aa, Artificial
  synthetic and modified peptide derived from human MUC1
APDTRPAPG

SEQ ID NO: 7 - protein, 10 aa, Artificial
  synthetic and modified peptide derived from human MUC1
APDTRPAPGS

SEQ ID NO: 8 - protein, 10 aa, Artificial
  synthetic and modified peptide derived from human MUC1
APDTRPAPGS

SEQ ID NO: 9 - protein, 9 aa, Artificial
  synthetic and modified peptide derived from human MUC1
PDTRPAPGS

SEQ ID NO: 10 - protein, 9 aa, Artificial
  synthetic and modified peptide derived from human MUC1
PDTRPAPGS

SEQ ID NO: 11 - protein, 10 aa, Artificial
  synthetic and modified peptide derived from human MUC1
PDTRPAPGST

SEQ ID NO: 12 - protein, 10 aa, Artificial
  synthetic and modified peptide derived from human MUC1
PDTRPAPGST

SEQ ID NO: 13 - protein, 100 aa, Homo sapiens
  MISC_FEATURE: fragment of digested MUC1
GVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAH

SEQ ID NO: 14 - protein, 61 aa, Homo sapiens
  MISC_FEATURE: Fragment of digested MUC1
AHGVTSAPESRPAPGSTAPPAHGVTSAPESRPAPGSTAPPAHGVTSAPESRPAPGSTAPPA

SEQ ID NO: 15 - protein, 21 aa, Homo sapiens
  MISC_FEATURE: fragment of digested MUC1
AHGVTSAPDTRPAPGSTAPPA

SEQ ID NO: 16 - protein, 21 aa, Homo sapiens
  MISC_FEATURE: fragment of digested MUC1
AHGVTSAPESRPAPGSTAPPA

SEQ ID NO: 17 - protein, 21 aa, Homo sapiens
  MISC_FEATURE: Threonin position 5 is o-glycosylated
AHGVTSAPDTRPAPGSTAPPA

SEQ ID NO: 18 - protein, 21 aa, Homo sapiens
  MISC_FEATURE: Threonin position 10 is O-glycosylated
AHGVTSAPDTRPAPGSTAPPA

SEQ ID NO: 19 - protein, 21 aa, Homo sapiens
  MISC_FEATURE: Threonin position 17 is O-glycosylated
AHGVTSAPDTRPAPGSTAPPA

SEQ ID NO: 20 - protein, 21 aa, Homo sapiens
  MISC_FEATURE: Threonin position 5 is O-glycosylated
AHGVTSAPESRPAPGSTAPPA

SEQ ID NO: 21 - protein, 21 aa, Homo sapiens
  MISC_FEATURE: Serin position 10 is O-glycosylated
AHGVTSAPESRPAPGSTAPPA

SEQ ID NO: 22 - protein, 21 aa, Homo sapiens
  MISC_FEATURE: Threonin position 17 is O-glycosylated
AHGVTSAPESRPAPGSTAPPA

SEQ ID NO: 23 - protein, 21 aa, Homo sapiens
  MISC_FEATURE: Serin position 6 is O-glycosylated
AHGVTSAPESRPAPGSTAPPA

SEQ ID NO: 24 - protein, 8 aa, Homo sapiens
  MISC_FEATURE: fragment of MUC1
APESRPAP

SEQ ID NO: 25 - protein, 8 aa, Homo sapiens
  MISC_FEATURE: fragment of MUC1
TSAPESRP

SEQ ID NO: 26 - protein, 9 aa, Homo sapiens
  MISC_FEATURE: fragment of MUC1
APESRPAPG

SEQ ID NO: 27 - protein, 9 aa, Homo sapiens
  MISC_FEATURE: fragment of MUC1
PESRPAPGS

SEQ ID NO: 28 - protein, 10 aa, Homo sapiens
  MISC_FEATURE: fragment of MUC1
STAPPAHGVT

SEQ ID NO: 29 - protein, 10 aa, Homo sapiens
  MISC_FEATURE: fragment of MUC1
TAPPAHGVTS

SEQ ID NO: 30 - protein, 10 aa, Homo sapiens
  MISC_FEATURE: fragment of MUC1
SAPESRPAPG

SEQ ID NO: 31 - protein, 10 aa, Homo sapiens
  MISC_FEATURE: fragment of MUC1
APESRPAPGS

SEQ ID NO: 32 - protein, 10 aa, Homo sapiens
  MISC_FEATURE: fragment of MUC1
PESRPAPGST

SEQ ID NO: 33 - protein, 11 aa, Homo sapiens
  MISC_FEATURE: fragment of MUC1
SAPESRPAPGS

SEQ ID NO: 34 - protein, 12 aa, Homo sapiens
  MISC_FEATURE: fragment of MUC1
GVTSAPESRPAP

SEQ ID NO: 35 - protein, 12 aa, Homo sapiens
  MISC_FEATURE: fragment of MUC1
VTSAPESRPAPG

SEQ ID NO: 36 - protein, 13 aa, Homo sapiens
  MISC_FEATURE: fragment of MUC1
PESRPAPGSTAPP

SEQ ID NO: 37 - protein, 16 aa, Homo sapiens
  MISC_FEATURE: fragment of MUC1
TSAPESRPAPGSTAPP

SEQ ID NO: 38 - protein, 16 aa, Homo sapiens
  MISC_FEATURE: fragment of MUC1
HGVTSAPESRPAPGST

SEQ ID NO: 39 - protein, 17 aa, Homo sapiens
  MISC_FEATURE: fragment of MUC1
SAPESRPAPGSTAPPAH

SEQ ID NO: 40 - protein, 18 aa, Homo sapiens
  MISC_FEATURE: fragment of MUC1
AHGVTSAPESRPAPGSTA

SEQ ID NO: 41 - protein, 17 aa, Homo sapiens
  MISC_FEATURE: fragment of MUC1
VTSAPESRPAGSTAPPA

SEQ ID NO: 42 - protein, 19 aa, Homo sapiens
  MISC_FEATURE: fragment of MUC1
SAPESRPAPGSTAPPAHGV

SEQ ID NO: 43 - protein, 21 aa, Homo sapiens
  MISC_FEATURE: fragment of MUC1
VTSAPDTRPAPGSTAPPAHGV

SEQ ID NO: 44 - protein, 25 aa, Homo sapiens
  MISC_FEATURE: fragment of MUC1
TAPPAHGVTSAPESRPAPGSTAPPA

SEQ ID NO: 45 - protein, 25 aa, Homo sapiens
  MISC_FEATURE: fragment of MUC1
TSAPESRPAPGSTAPPAHGTSAPES

SEQ ID NO: 46 - protein, 9 aa, Homo sapiens
  MISC_FEATURE: fragment of MUC1
SAPESRPAP

SEQ ID NO: 47 - protein, 10 aa, Homo sapiens
  MISC_FEATURE: fragment of MUC 1
SAPESRPAPG

SEQ ID NO: 48 - protein, 9 aa, Homo sapiens
  MISC_FEATURE: fragment of MUC1
TSAPDTRPA

SEQ ID NO: 49 - protein, 8 aa, Homo sapiens
  MISC_FEATURE: fragment of MUC1
PGSTAPPA

SEQ ID NO: 50 - protein, 8 aa, Homo sapiens
  MISC_FEATURE: fragment of MUC1
RPAPGSTA

SEQ ID NO: 51 - protein, 8 aa, Homo sapiens
  MISC_FEATURE: fragment of MUC1
SAPDTRPA

SEQ ID NO: 52 - protein, 9 aa, Homo sapiens
  MISC_FEATURE: fragment of MUC1
SAPDTRPAP

SEQ ID NO: 53 - protein, 10 aa, Homo sapiens
  MISC_FEATURE: fragment of MUC1
SAPDTRPAPG

SEQ ID NO: 54 - protein, 8 aa, Homo sapiens
  MISC_FEATURE: fragment of MUC1
SAPESRPA

SEQ ID NO: 55 - protein, 9 aa, Homo sapiens
  MISC_FEATURE: fragment of MUC1
SRPAPGSTA

SEQ ID NO: 56 - protein, 10 aa, Homo sapiens
  MISC_FEATURE: fragment of MUC1
VTSAPESRPA

SEQ ID NO: 57 - protein, 10 aa, Homo sapiens
  MISC_FEATURE: fragment of MUC1
PAPGSTAPPA

SEQ ID NO: 58 - protein, 9 aa, Homo sapiens
  MISC_FEATURE: fragment of MUC1
TSAPESRPA

SEQ ID NO: 59 - protein, 9 aa, Homo sapiens
  MISC_FEATURE: fragment of MUC1
APGSTAPPA

SEQ ID NO: 60 - protein, 9 aa, Homo sapiens
  MISC_FEATURE: fragment of MUC1
PAPGSTAPP

SEQ ID NO: 61 - protein, 9 aa, Homo sapiens
  MISC_FEATURE: fragment of MUC1
RPAPGSTAP

SEQ ID NO: 62 - protein, 9 aa, Homo sapiens
  MISC_FEATURE: fragment of MUC1
TRPAPGSTA

SEQ ID NO: 63 - protein, 9 aa, Homo sapiens
  MISC_FEATURE: fragment of MUC1
GVTSAPDTR

SEQ ID NO: 64 - protein, 9 aa, Homo sapiens
  MISC_FEATURE: fragment of MUC1
GVTSAPESR

SEQ ID NO: 65 - protein, 10 aa, Homo sapiens
  MISC_FEATURE: fragment of MUC1
RPAPGSTAPP

SEQ ID NO: 66 - protein, 10 aa, Homo sapiens
  MISC_FEATURE: fragment of MUC1
AHGVTSAPES

SEQ ID NO: 67 - protein, 9 aa, Homo sapiens
  MISC_FEATURE: fragment of MUC1
ESRPAPGST

SEQ ID NO: 68 - protein, 10 aa, Homo sapiens
  MISC_FEATURE: fragment of MUC1
ESRPAPGSTA

SEQ ID NO: 69 - protein, 9 aa, Homo sapiens
  MISC_FEATURE: fragment of MUC1
AHGVTSAPE

SEQ ID NO: 70 - protein, 9 aa, Homo sapiens
  MISC_FEATURE: fragment of MUC1
APDTRPAPG

SEQ ID NO: 71 - protein, 10 aa, Homo sapiens
  MISC_FEATURE: fragment of MUC1
GVTSAPDTRP

SEQ ID NO: 72 - protein, 10 aa, Homo sapiens
  MISC_FEATURE: fragment of MUC1
GVTSAPESRP

SEQ ID NO: 73 - protein, 9 aa, Homo sapiens
  MISC_FEATURE: fragment of MUC1
APESRPAPG

SEQ ID NO: 74 - protein, 10 aa, Homo sapiens
  MISC_FEATURE: fragment of MUC1
APESRPAPGS

SEQ ID NO: 75 - protein, 9 aa, Homo sapiens
  MISC_FEATURE: fragment of MUC1
HGVTSAPES

SEQ ID NO: 76 - protein, 10 aa, Homo sapiens
  MISC_FEATURE: fragment of MUC1
PESRPAPGST

SEQ ID NO: 77 - protein, 10 aa, Homo sapiens
  MISC_FEATURE: fragment of MUC1
SRPAPGSTAP

SEQ ID NO: 78 - protein, 10 aa, Homo sapiens
  MISC_FEATURE: fragment of MUC1
HGVTSAPDTR

SEQ ID NO: 79 - protein, 10 aa, Homo sapiens
  MISC_FEATURE: fragment of MUC1
HGVTSAPESR

SEQ ID NO: 80 - protein, 10 aa, Homo sapiens
  MISC_FEATURE: fragment of MUC1
PDTRPAPGST

SEQ ID NO: 81 - protein, 10 aa, Homo sapiens
  MISC_FEATURE: fragment of MUC1
TRPAPGSTAP

SEQ ID NO: 82 - protein, 8 aa, Homo sapiens
  MISC_FEATURE: fragment of MUC1
GVTSAPDT

SEQ ID NO: 83 - protein, 8 aa, Homo sapiens
  MISC_FEATURE: fragment of MUC1
GVTSAPES

SEQ ID NO: 84 - protein, 8 aa, Homo sapiens
  MISC_FEATURE: fragment of MUC1
HGVTSAPD

SEQ ID NO: 85 - protein, 8 aa, Homo sapiens
  MISC_FEATURE: fragment of MUC1
PAPGSTAP

SEQ ID NO: 86 - protein, 8 aa, Homo sapiens
  MISC_FEATURE: fragment of MUC1
PDTRPAPG

SEQ ID NO: 87 - protein, 9 aa, Homo sapiens
  MISC_FEATURE: fragment of MUC1
PDTRPAPGS

SEQ ID NO: 88 - protein, 8 aa, Homo sapiens
  MISC_FEATURE: fragment of MUC1
PESRPAPG

SEQ ID NO: 89 - protein, 9 aa, Homo sapiens
  MISC_FEATURE: fragment of MUC1
PESRPAPGS

SEQ ID NO: 90 - protein, 8 aa, Homo sapiens
  MISC_FEATURE: fragment of MUC1
SRPAPGST

SEQ ID NO: 91 - protein, 8 aa, Homo sapiens
  MISC_FEATURE: fragment of MUC1
TRPAPGST

SEQ ID NO: 92 - protein, 8 aa, Homo sapiens
  MISC_FEATURE: fragment of MUC1
TSAPDTRP

SEQ ID NO: 93 - protein, 8 aa, Homo sapiens
  MISC_FEATURE: fragment of MUC1
TSAPESRP

SEQ ID NO: 94 - protein, 8 aa, Homo sapiens
  MISC_FEATURE: fragment of MUC1
VTSAPDTR

SEQ ID NO: 95 - protein, 8 aa, Homo sapiens
  MISC_FEATURE: fragment of MUC1
APGSTAPP

SEQ ID NO: 96 - protein, 11 aa, Homo sapiens
  MISC_FEATURE: fragment of MUC1
AHGVTSAPDTR

SEQ ID NO: 97 - protein, 11 aa, Homo sapiens
  MISC_FEATURE: fragment of MUC1
AHGVTSAPESR

SEQ ID NO: 98 - protein, 8 aa, Homo sapiens
  MISC_FEATURE: fragment of MUC1
APDTRPAP

SEQ ID NO: 99 - protein, 11 aa, Homo sapiens
  MISC_FEATURE: fragment of MUC1
APDTRPAPGST

SEQ ID NO: 100 - protein, 8 aa, Homo sapiens
  MISC_FEATURE: fragment of MUC1
APESRPAP

SEQ ID NO: 101 - protein, 11 aa, Homo sapiens
  MISC_FEATURE: fragment of MUC1
APESRPAPGST

SEQ ID NO: 102 - protein, 8 aa, Homo sapiens
  MISC_FEATURE: fragment of MUC1
DTRPAPGS

SEQ ID NO: 103 - protein, 11 aa, Homo sapiens
  MISC_FEATURE: fragment of MUC1
DTRPAPGSTAP

SEQ ID NO: 104 - protein, 8 aa, Homo sapiens
  MISC_FEATURE: fragment of MUC1
ESRPAPGS

SEQ ID NO: 105 - protein, 11 aa, Homo sapiens
  MISC_FEATURE: fragment of MUC1
ESRPAPGSTAP

SEQ ID NO: 106 - protein, 11 aa, Homo sapiens
  MISC_FEATURE: fragment of MUC1
HGVTSAPESRP

SEQ ID NO: 107 - protein, 11 aa, Homo sapiens
  MISC_FEATURE: fragment of MUC1
PDTRPAPGSTA

SEQ ID NO: 108 - protein, 11 aa, Homo sapiens
  MISC_FEATURE: fragment of MUC1
PESRPAPGSTA

SEQ ID NO: 109 - protein, 11 aa, Homo sapiens
  MISC_FEATURE: fragment of MUC1
RPAPGSTAPPA

SEQ ID NO: 110 - protein, 11 aa, Homo sapiens
  MISC_FEATURE: fragment of MUC1
SAPDTRPAPGS

SEQ ID NO: 111 - protein, 11 aa, Homo sapiens
  MISC_FEATURE: fragment of MUC1
SAPESRPAPGS

SEQ ID NO: 112 - protein, 11 aa, Homo sapiens
  MISC_FEATURE: fragment of MUC1
SRPAPGSTAPP

SEQ ID NO: 113 - protein, 11 aa, Homo sapiens
  MISC_FEATURE: fragment of MUC1
TRPAPGSTAPP

SEQ ID NO: 114 - protein, 11 aa, Homo sapiens
  MISC_FEATURE: fragment of MUC1
TSAPDTRPAPG

SEQ ID NO: 115 - protein, 11 aa, Homo sapiens
  MISC_FEATURE: fragment of MUC1
TSAPESRPAPG

SEQ ID NO: 116 - protein, 11 aa, Homo sapiens
  MISC_FEATURE: fragment of MUC1
VTSAPDTRPAP

SEQ ID NO: 117 - protein, 11 aa, Homo sapiens
  MISC_FEATURE: fragment of MUC1
VTSAPESRPAP

SEQ ID NO: 118 - protein, 8 aa, Homo sapiens
  MISC_FEATURE: fragment of MUC1
VTSAPESR

SEQ ID NO: 119 - protein, 60 aa, Homo sapiens
  MISC_FEATURE: fragment of MUC1
GVTSAPDTRPAPGSTAPPAHGVTSAPDTRPAPGSTAPPAHGVTSAPDTRPAPGSTAPPAH

SEQ ID NO: 120 - protein, 30 aa, Homo sapiens
  MISC_FEATURE: fragment of MUC1
SAPDTRPAPGSTAPPAHGVTSAPDTRPAPG

SEQ ID NO: 121 - protein, 25 aa, Homo sapiens
  MISC_FEATURE: fargment of MUC1
RPAPGSTAPPAHGVTSAPDTRPAPG

SEQ ID NO: 122 - protein, 20 aa, Homo sapiens
  MISC_FEATURE: fragment of MUC1
RPAPGSTAPPAHGVTSAPDT

SEQ ID NO: 123 - protein, 15 aa, Homo sapiens
  MISC_FEATURE: fragment of MUC1
RPAPGSTAPPAHGVT

SEQ ID NO: 124 - protein, 10 aa, Homo sapiens
  MISC_FEATURE: fragment of MUC1
STAPPAHGVT

SEQ ID NO: 125 - protein, 27 aa, Homo sapiens
  MISC_FEATURE: fragment of MUC1
STAPPAHGVTSAPDTRPAPGSTAPPAH

SEQ ID NO: 126 - protein, 10 aa, Homo sapiens
  MISC_FEATURE: fragment of MUC1
STAPPAHGVT

SEQ ID NO: 127 - protein, 7 aa, Homo sapiens
  MISC_FEATURE: fragment of MUC1
STAPPAH

SEQ ID NO: 128 - protein, 20 aa, Homo sapiens
  MISC_FEATURE: fragment of MUC1
GVTSAPDTRPAPGSTAPPAH

SEQ ID NO: 129 - protein, 17 aa, Homo sapiens
  MISC_FEATURE: fragment of MUC1
SAPDTRPAPGSTAPPAH

SEQ ID NO: 130 - protein, 12 aa, Homo sapiens
  MISC_FEATURE: fragment of MUC1
RPAPGSTAPPAH

SEQ ID NO: 131 - protein, 33 aa, Homo sapiens
  MISC_FEATURE: fragment of MUC1
GVTSAPDTRPAPGSTAPPAHGVTSAPDTRPAPG

SEQ ID NO: 132 - protein, 13 aa, Homo sapiens
  MISC_FEATURE: fragment of MUC1
GVTSAPDTRPAPG

SEQ ID NO: 133 - protein, 10 aa, Homo sapiens
  MISC_FEATURE: fragment of MUC1
SAPDTRPAPG

SEQ ID NO: 134 - protein, 21 aa, Artificial
  synthetic and modified peptide derived from human MUC1
AHGVTSAPDTRPAPGSTAPPA

SEQ ID NO: 135 - protein, 21 aa, Artificial
  synthetic and modified peptide derived from human MUC1
AHGVTSAPDTRPAPGSTAPPA

SEQ ID NO: 136 - protein, 21 aa, Artificial
  synthetic and modified peptide derived from human MUC1
AHGVTSAPDTRPAPGSTAPPA

SEQ ID NO: 137 - protein, 21 aa, Artificial
  synthetic and modified peptide derived from human MUC1
AHGVTSAPDTRPAPGSTAPPA

SEQ ID NO: 138 - protein, 21 aa, Artificial
  synthetic and modified peptide derived from human MUC1
AHGVTSAPDTRPAPGSTAPPA

SEQ ID NO: 139 - protein, 10 aa, Artificial
  synthetic and modified peptide derived from human MUC1
FLPSDCFPSV

SEQ ID NO: 140 - protein, 10 aa, Artificial
  synthetic and modified peptide derived from human MUC1
STAPPAHGVT

SEQ ID NO: 141 - protein, 10 aa, Artificial
  synthetic and modified peptide derived from human MUC1
STAPPAHGVT

SEQ ID NO: 142 - protein, 9 aa, Artificial
  synthetic and modified peptide derived from human MUC1
STAPPAHGV

SEQ ID NO: 143 - protein, 9 aa, Artificial
  synthetic and modified peptide derived from human MUC1
STAPPAHGV

SEQ ID NO: 144 - protein, 10 aa, Artificial
  synthetic and modified peptide derived from human MUC1
SAPDTRPAPG

SEQ ID NO: 145 - protein, 10 aa, Artificial
  synthetic and modified peptide derived from human MUC1
SAPDTRPAPG

SEQ ID NO: 146 - protein, 10 aa, Artificial
  synthetic and modified peptide derived from human MUC1
SAPDTRPAPG

SEQ ID NO: 147 - protein, 10 aa, Artificial
  synthetic and modified peptide derived from human MUC1
SAPDTRPAPG

SEQ ID NO: 148 - protein, 10 aa, Artificial
  synthetic and modified peptide derived from human MUC1
SAPDTRPAPG

SEQ ID NO: 149 - protein, 10 aa, Artificial
  synthetic and modified peptide derived from human MUC1
APPAHGVTSA

SEQ ID NO: 150 - protein, 10 aa, Artificial
  synthetic and modified peptide derived from human MUC1
APPAHGVTSA

SEQ ID NO: 151 - protein, 9 aa, Artificial
  synthetic and modified peptide derived from human MUC1
GVTSAPESR

SEQ ID NO: 152 - protein, 9 aa, Artificial
  synthetic and modified peptide derived from human MUC1
GVTSAPESR

SEQ ID NO: 153 - protein, 9 aa, Artificial
  synthetic and modified peptide derived from human MUC1
GVTSAPDTR

SEQ ID NO: 154 - protein, 9 aa, Artificial
  synthetic and modified peptide derived from human MUC1
GVTSAPDTR